(19) United States
(12) Patent Application Publication
(10) Pub. No.: US 2022/0249620 A1
(43) Pub. Date: Aug. 11, 2022

(54) COMPOSITION COMPRISING RECOMBINANT PARATHYROID HORMONE FOR HEALING AFTER ROTATOR CUFF REPAIR

(71) Applicant: Seoul National University Hospital, Seoul (KR)

(72) Inventors: Joo Han Oh, Seongnam-Si (KR); Sung-Min Rhee, Seoul (KR)

(21) Appl. No.: 17/599,744

(22) PCT Filed: Mar. 27, 2020
(86) PCT No.: PCT/KR2020/004260

§ 371 Date: Apr. 20, 2022

(30) Foreign Application Priority Data
Mar. 29, 2019 (KR) 10-2019-0036716

(51) Int. Cl. A61K 38/29; A61K 9/00; A61P 19/00

(52) U.S. Cl. CPC A61K 38/29; A61P 19/00; A61K 9/0019

(57) ABSTRACT

The present disclosure relates to a composition for healing after rotator cuff repair, more specifically to a composition containing teriparatide as an active ingredient for healing tears of a suture site after rotator cuff repair, and the composition exhibits a tendon-to-bone healing effect when administered to patients, especially those with a tear size larger than 2 cm, for treatment of rotator cuff and is effective in lowering re-tearing rate, and thus can lead to improved healing after rotator cuff repair.

### TECHNICAL FIELD

The present disclosure discloses a composition for healing of tear at the suture site after rotator cuff repair, which contains teriparatide, an isomer thereof, a pharmaceutically acceptable salt thereof, a hydrate thereof or a solvate thereof as an active ingredient.

### BACKGROUND ART

The rapid development of surgical techniques for arthroscopic rotator cuff repair over the past 10 years has led to satisfactory anatomical and functional results. However, the high retear rate of 39.4-94%, particularly for patients with a large tear size, is a problem (Chung S W, Kim J Y, Kim M H, Kim S H, Oh J H. Arthroscopic repair of massive rotator cuff tears: outcome and analysis of factors associated with healing failure or poor postoperative function. The American journal of sports medicine. 2013; 41: 1674-1683; Galatz L M, Ball C M, Teefey S A, Middleton W D, Yamaguchi K. The outcome and repair integrity of completely arthroscopically repaired large and massive rotator cuff tears. The Journal of bone and joint surgery. American volume. 2004; 86-A: 219-224). Therefore, many researches have been conducted to find prognostic factors and reduce retear rate (Chung S W, Oh J H, Gong H S, Kim J Y, Kim S H. Factors affecting rotator cuff healing after arthroscopic repair: osteoporosis as one of the independent risk factors. The American journal of sports medicine. 2011; 39: 2099-2107.), but further researches are necessary. Recently, use of various biological augmentation agents including stem cells (Oh J H, Chung S W, Kim S H, Chung J Y, Kim J Y. 2013 Neer Award: Effect of the adipose-derived stem cell for the improvement of fatty degeneration and rotator cuff healing in rabbit model. Journal of shoulder and elbow surgery. 2014; 23: 445-455), platelet-rich plasma (Chung S W, Song B W, Kim Y H, Park K U, Oh J H. Effect of platelet-rich plasma and porcine dermal collagen graft augmentation for rotator cuff healing in a rabbit model. The American journal of sports medicine. 2013; 41: 2909-2918; Saltzman B M, Jain A, Campbell K A, et al. Does the Use of Platelet-Rich Plasma at the Time of Surgery Improve Clinical Outcomes in Arthroscopic Rotator Cuff Repair When Compared With Control Cohorts? A Systematic Review of Meta-analyses. Arthroscopy: the journal of arthroscopic & related surgery: official publication of the Arthroscopy Association of North America and the International Arthroscopy Association. 2016; 32: 906-918.) or fibroblasts (Kwon J, Kim Y H, Rhee S M, et al. Effects of Allogenic Dermal Fibroblasts on Rotator Cuff Healing in a Rabbit Model of Chronic Tear. The American journal of sports medicine. 2018; 46: 1901-1908.) at the tendon-to-bone junction site has been conducted for enhancement of healing after arthroscopic rotator cuff repair. Although results of improving the effect of rotator cuff repair by administering some biological augmentation agents directly to the suture site have been reported, methods for systemic treatment are still insufficient.

Teriparatide, which is a recombinant human parathyroid hormone (rhPTH), improves bone mineral density by stimulating bone formation. In addition, it has been found out that systemic treatment with teriparatide has an independent effect also in tendon-to-bone healing of the rotator cuff repair site (Duchman K R, Goetz J E, Uribe B U, et al. Delayed administration of recombinant human parathyroid hormone improves early biomechanical strength in a rat rotator cuff repair model. Journal of shoulder and elbow surgery. 2016; 25: 1280-1287; Bi F, Shi Z, Jiang S, Guo P, Yan S. Intermittently administered parathyroid hormone [1-34] promotes tendon-bone healing in a rat model. International journal of molecular sciences. 2014; 15: 17366-17379; Lee D J, Southgate R D, Farhat Y M, et al. Parathyroid hormone 1-34 enhances extracellular matrix deposition and organization during flexor tendon repair. Journal of orthopaedic research: official publication of the Orthopaedic Research Society. 2015; 33: 17-24.). Hettrich et al. reported that treatment with rhPTH in a rat rotator cuff tear model increases bone and fibrocartilage formation (Hettrich C M, Beamer B S, Bedi A, et al. The effect of rhPTH on the healing of tendon to bone in a rat model. Journal of orthopaedic research: official publication of the Orthopaedic Research Society. 2012; 30: 769-774.). Although there have been some researches on rat rotator cuff tear models, clinical researches on human have not been reported yet. In addition, according to the research by Hettrich et al., biomechanical characteristics were not improved after the administration of rhPTH, which may be due to excessive vascularization that may cause adverse effects on mechanical characteristics in the early stage after surgery.

Furthermore, it is known that excessive or long-term administration of teriparatide causes side effects. Through this, it can be seen that the time, period, dosage, etc. of teriparatide administration are important. But, researches have not been conducted on the time, period, dosage, etc. of teriparatide administration for patients with rotator cuff tear.

Therefore, the inventors of the present disclosure have conducted researches to identify the effect of the recombinant parathyroid hormone, teriparatide, on patients who have received rotator cuff repair and the time, period, dosage, etc. of administration, and have completed the present disclosure.

### DISCLOSURE

### Technical Problem

In an aspect, the present disclosure is directed to providing a composition for healing after rotator cuff repair, wherein the composition contains teriparatide, an isomer thereof, a pharmaceutically acceptable salt thereof, a hydrate thereof or a solvate thereof as an active ingredient, the healing after rotator cuff repair is healing of tear at the suture site after rotator cuff repair, and the composition is administered to a patient having a rotator cuff tear size of larger than 2 cm at a dosage of 0.066-1.67 μg/kg/day after rotator cuff repair.

In another aspect, the present disclosure is directed to providing a kit for healing after rotator cuff repair, which includes the composition.

### Technical Solution

In an aspect, the present disclosure provides a composition for healing after rotator cuff repair, wherein the composition contains teriparatide, an isomer thereof, a pharmaceutically acceptable salt thereof, a hydrate thereof or a solvate thereof as an active ingredient, the healing after rotator cuff repair is healing of tear at the suture site after rotator cuff repair, and the composition is administered to a patient having a rotator cuff tear size of larger than 2 cm at a dosage of 0.066-1.67 μg/kg/day after rotator cuff repair.

In another aspect, the present disclosure provides a kit for healing after rotator cuff repair, which includes the composition.

### Advantageous Effects

The present disclosure relates to a composition for healing after rotator cuff repair, more specifically to a composition containing teriparatide as an active ingredient for healing of tear at the suture site after rotator cuff repair. The composition exhibits a tendon-to-bone healing effect when administered to patients, especially those with a tear size larger than 2 cm, for treatment of rotator cuff tear and is effective in lowering retear rate, and thus can lead to improved healing after rotator cuff repair.

### BRIEF DESCRIPTION OF DRAWINGS

FIG. 1 schematically describes a process of selecting a teriparatide administration group (test group, n=31) and a teriparatide non-administration group (control group, n=124) according to the present disclosure.

### BEST MODE

Hereinafter, the present disclosure is described in detail.

In an aspect, the present disclosure provides a composition for healing after rotator cuff repair, wherein the composition contains teriparatide, an isomer thereof, a pharmaceutically acceptable salt thereof, a hydrate thereof or a solvate thereof as an active ingredient, the healing after rotator cuff repair is healing of tear at the suture site after rotator cuff repair, and the composition is administered to a patient having a rotator cuff tear size of larger than 2 cm at a dosage of 0.066-1.67 μg/kg/day after rotator cuff repair.

The teriparatide is a recombinant human parathyroid hormone (rhPTH) and may have a structure of Chemical Formula 1. Teriparatide is identical to a portion of human parathyroid hormone (PTH) and intermittent use activate osteoblasts more than osteoclasts, which leads to improvement of bone mineral density (BMD). In the present disclosure, the teriparatide may be teriparatide (Forteo®) purchased from Eli Lilly (Indianapolis, Ind.), although not being limited thereto.

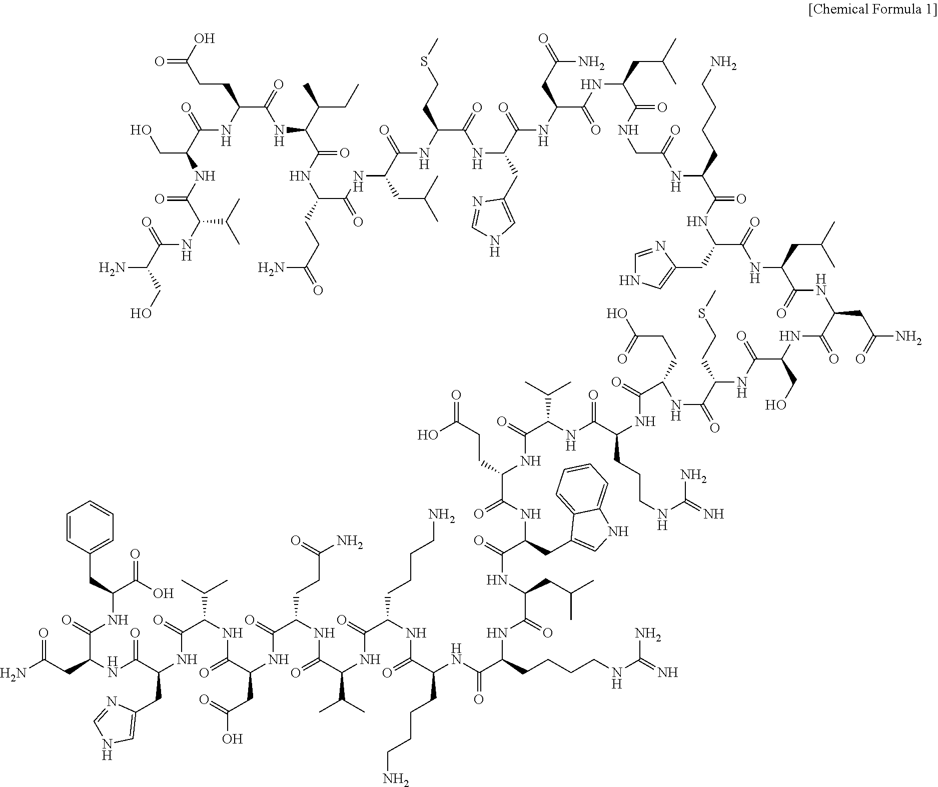

In an aspect of the present disclosure, “pharmaceutically acceptable” means that use of a general medicinal dosage avoids a significant toxic effect and thus can be approved or is approved as appropriate in application to animals, particularly to human, by the government or corresponding regulations organizations, or is listed in the pharmacopeia or regarded as described in general pharmacopeias.

In an aspect of the present disclosure, the “pharmaceutically acceptable salt” means a salt according to an aspect of the present disclosure that is pharmaceutically acceptable and has the desired pharmacological activity of the parent. The salt may include: (1) an acid addition salt formed with an inorganic acid such as hydrochloric acid, hydrobromic acid, sulfuric acid, nitric acid, phosphoric acid, etc. or with an organic acid such as acetic acid, propionic acid, hexanoic acid, cyclopentylpropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl) benzoic acid, cinnamic acid, mandelic acid, methanesulfonic acid, ethanesulfonic acid, 1,2-ethanedisulfonic acid, 2-hydroxyehtanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2,2,2]-oct-2-ene-1-carboxylic acid, glucoheptonic acid, 3-phenylpropionic acid, trimethylacetic acid, tert-butylacetic acid, lauryl sulfuric acid, gluconic acid, glutamic acid, hydroxynaphthoic acid, salicylic acid, stearic acid or muconic acid; or (2) a salt formed as an acidic proton present in the parent compound is substituted.

In an aspect of the present disclosure, the “isomer” particularly includes not only optical isomers (e.g., essentially pure enantiomers, essentially pure diastereomers or a mixture thereof) but also conformation isomers (i.e., isomers having a different angle in at least one chemical bond), position isomers (particularly, tautomers) or geometric isomers (e.g., cis-trans isomers).

In an aspect of the present disclosure, the “hydrate” means a compound to which water is bound and is used in its broadest concept including an inclusion compound having no chemical binding force between water and the compound.

In an aspect of the present disclosure, the “solvate” means a higher-order compound formed from a molecule or ion of a solute and a molecule or ion of a solvent.

The composition may contain the active ingredient teriparatide at a concentration of 1.66-41.67 μg/mL, specifically 1.66 μg/mL or higher, 2 μg/mL or higher, 3 μg/mL or higher, 4 μg/mL or higher, 5 μg/mL or higher, 6 μg/mL or higher, 7 μg/mL or higher, 7.2 μg/mL or higher, 7.4 μg/mL or higher, 7.6 μg/mL or higher, 7.8 μg/mL or higher, 8 μg/mL or higher, 8.1 μg/mL or higher, 8.15 μg/mL or higher, 8.2 μg/mL or higher, 8.25 μg/mL or higher, 8.3 μg/mL or higher, 8.5 μg/mL or higher, 9 μg/mL or higher, 10 μg/mL or higher, 15 μg/mL or higher, 20 μg/mL or higher, 25 μg/mL or higher, 30 μg/mL or higher, 35 μg/mL or higher or 40 μg/mL or higher and 41.67 μg/mL or lower, 40 μg/mL or lower, 30 μg/mL or lower, 25 μg/mL or lower, 20 μg/mL or lower, 15 μg/mL or lower, 10 μg/mL or lower, 9.8 μg/mL or lower, 9.6 μg/mL or lower, 9.4 μg/mL or lower, 9.2 μg/mL or lower, 9 μg/mL or lower, 8.9 μg/mL or lower, 8.8 μg/mL or lower, 8.7 μg/mL or lower, 8.6 μg/mL or lower, 8.5 μg/mL or lower, 8.4 μg/mL or lower, 8.3 μg/mL or lower, 8 μg/mL or lower, 6 μg/mL or lower, 4 μg/mL or lower or 2 μg/mL or lower, based on the total volume of the composition, although not being limited thereto.

In an aspect of the present disclosure, wound may be selected from a group consisting of non-healing traumatic wound, radiation-induced tissue destruction, abrasion, laceration, avulsion, penetration wound, gunshot wound, incision, burn, frostbite, skin ulcer, skin dryness, keratoderma, fissure, burst, dermatitis, surgical wound, vascular wound, bruise, corneal wound, bedsore, decubitus, chronic ulcer, post-surgical suture site, spinal wound, gynecological wound, chemical wound and acne, specifically wound of a suture site after rotator cuff surgery, more specifically wound of a suture site after rotator cuff repair, further more specifically tear at a suture site after rotator cuff repair, further more specifically tear or rupture at a suture site larger than 2 cm after rotator cuff repair, although not being limited thereto. The healing after rotator cuff repair may be specifically healing of tear at a suture site after rotator cuff repair, more specifically healing of tear or rupture at a suture site larger than 2 cm after rotator cuff repair, although not being limited thereto.

In an aspect of the present disclosure, the tear may be ripping, bursting, tearing loose or splitting, specifically, ripping, bursting, tearing loose or splitting of a suture site after rotator cuff repair, although not being limited thereto.

The composition may be administered to a patient who has received rotator cuff repair. Specifically, it may be administered to a patient who has a rotator cuff tear size larger than 2 cm after rotator cuff repair.

The composition may be administered to a patient who has a rotator cuff tear size larger than 2 cm after rotator cuff repair at a dosage of 0.066-1.67 μg/kg/day. The administration dosage of the composition or the active ingredient teriparatide may vary depending on the age, sex and body weight of a subject to be treated, the particular disease or pathological condition to be treated, the severity of the disease or pathological condition, administration route and the discretion of a prescriber. The determination of the administration dosage based on these factors is within the level of those skilled in the art.

The administration dosage of the composition or the active ingredient teriparatide may be 0.066-1.67 μg/kg/day, specifically 0.166-0.667 μg/kg/day, more specifically 0.066 μg/kg/day or more, 0.08 μg/kg/day or more, 0.1 μg/kg/day or more, 0.11 μg/kg/day or more, 0.12 μg/kg/day or more, 0.13 μg/kg/day or more, 0.14 μg/kg/day or more, 0.15 μg/kg/day or more, 0.16 μg/kg/day or more, 0.17 μg/kg/day or more, 0.18 μg/kg/day or more, 0.19 μg/kg/day or more, 0.2 μg/kg/day or more, 0.21 μg/kg/day or more, 0.22 μg/kg/day or more, 0.23 μg/kg/day or more, 0.24 μg/kg/day or more, 0.25 μg/kg/day or more, 0.26 μg/kg/day or more, 0.27 μg/kg/day or more, 0.28 μg/kg/day or more, 0.29 μg/kg/day or more, 0.3 μg/kg/day or more, 0.31 μg/kg/day or more, 0.32 μg/kg/day or more, 0.33 μg/kg/day or more, 0.34 μg/kg/day or more, 0.35 μg/kg/day or more, 0.4 μg/kg/day or more, 0.5 μg/kg/day or more, 0.6 μg/kg/day or more, 0.7 μg/kg/day or more, 0.8 μg/kg/day or more, 0.9 μg/kg/day or more, 1 μg/kg/day or more, 1.2 μg/kg/day or more, 1.4 μg/kg/day or more or 1.6 μg/kg/day or more and 1.67 μg/kg/day or less, 1.5 μg/kg/day or less, 1.4 μg/kg/day or less, 1.3 μg/kg/day or less, 1.2 μg/kg/day or less, 1.1 μg/kg/day or less, 1 μg/kg/day or less, 0.9 μg/kg/day or less, 0.8 μg/kg/day or less, 0.7 μg/kg/day or less, 0.6 μg/kg/day or less, 0.5 μg/kg/day or less, 0.48 μg/kg/day or less, 0.46 μg/kg/day or less, 0.44 μg/kg/day or less, 0.42 μg/kg/day or less, 0.4 μg/kg/day or less, 0.39 μg/kg/day or less, 0.38 μg/kg/day or less, 0.37 μg/kg/day or less, 0.36 μg/kg/day or less, 0.35 μg/kg/day or less, 0.34 μg/kg/day or less, 0.33 μg/kg/day or less, 0.3 μg/kg/day or less, 0.2 μg/kg/day or less, 0.1 μg/kg/day or less, 0.09 μg/kg/day or less, 0.08 μg/kg/day or less or 0.07 μg/kg/day or less, although not being limited thereto.

Alternatively, the administration dosage of the composition or the active ingredient teriparatide may be 4-100 μg/day, specifically 4 μg/day or more, 5 μg/day or more, 6 μg/day or more, 7 μg/day or more, 8 μg/day or more, 9 μg/day or more, 10 μg/day or more, 10.5 μg/day or more, 11 μg/day or more, 11.5 μg/day or more, 12 μg/day or more, 12.5 μg/day or more, 13 μg/day or more, 13.5 μg/day or more, 14 μg/day or more, 14.5 μg/day or more, 15 μg/day or more, 15.5 μg/day or more, 16 μg/day or more, 16.5 μg/day or more, 17 μg/day or more, 17.5 μg/day or more, 18 μg/day or more, 18.5 μg/day or more, 19 μg/day or more, 19.5 μg/day or more, 20 μg/day or more, 21 μg/day or more, 22 μg/day or more, 23 μg/day or more, 24 μg/day or more, 25 μg/day or more, 26 μg/day or more, 27 μg/day or more, 28 μg/day or more, 29 μg/day or more, 30 μg/day or more, 32 μg/day or more, 34 μg/day or more, 36 μg/day or more, 38 μg/day or more, 40 μg/day or more, 50 μg/day or more, 60 μg/day or more, 70 μg/day or more, 80 μg/day or more or 90 μg/day or more and 100 μg/day or less, 90 μg/day or less, 80 μg/day or less, 70 μg/day or less, 60 μg/day or less, 50 μg/day or less, 40 μg/day or less, 38 μg/day or less, 36 μg/day or less, 34 μg/day or less, 32 μg/day or less, 30 μg/day or less, 29.5 μg/day or less, 29 μg/day or less, 28.5 μg/day or less, 28 μg/day or less, 27.5 μg/day or less, 27 μg/day or less, 26.5 μg/day or less, 26 μg/day or less, 25.5 μg/day or less, 25 μg/day or less, 24.5 μg/day or less, 24 μg/day or less, 23.5 μg/day or less, 23 μg/day or less, 22.5 μg/day or less, 22 μg/day or less, 21.5 μg/day or less, 21 μg/day or less, 20.5 μg/day or less, 20 μg/day or less, 19 μg/day or less, 18 μg/day or less, 17 μg/day or less, 16 μg/day or less, 15 μg/day or less, 14 μg/day or less, 13 μg/day or less, 12 μg/day or less, 11 μg/day or less, 10 μg/day or less, 8 μg/day or less, 6 μg/day or less, 4 μg/day or less, 2 μg/day or less or 1 μg/day or less, based on an adult weighing 60 kg, although not being limited thereto.

The composition may be administered for 2-24 weeks, specifically for 1-4 months, after rotator cuff repair. Specifically, the composition may be administered for 2 weeks or longer, 3 weeks or longer, 4 weeks or longer, 5 weeks or longer, 6 weeks or longer, 7 weeks or longer, 8 weeks or longer, 9 weeks or longer, 10 weeks or longer, 11 weeks or longer, 12 weeks or longer, 13 weeks or longer, 14 weeks or longer, 15 weeks or longer, 16 weeks or longer, 17 weeks or longer, 18 weeks or longer, 19 weeks or longer, 20 weeks or longer, 21 weeks or longer, 22 weeks or longer or 23 weeks or longer and 24 weeks or shorter, 23 weeks or shorter, 22 weeks or shorter, 21 weeks or shorter, 20 weeks or shorter, 19 weeks or shorter, 18 weeks or shorter, 17 weeks or shorter, 16 weeks or shorter, 15 weeks or shorter, 14 weeks or shorter, 13 weeks or shorter, 12 weeks or shorter, 11 weeks or shorter, 10 weeks or shorter, 9 weeks or shorter, 8 weeks or shorter, 7 weeks or shorter, 6 weeks or shorter, 5 weeks or shorter, 4 weeks or shorter or 3 weeks or shorter, after rotator cuff repair. Alternatively, the composition may be administered for 1 month or longer, 2 months or longer, 3 months or longer, 4 months or longer or 5 months or longer and 6 months or shorter, 5 months or shorter, 4 months or shorter, 3 months or shorter or 2 months or shorter, after rotator cuff repair, although not being limited thereto. Teriparatide, which is the active ingredient of composition, may have side effects such as nausea, vomiting, itchiness, muscle spasm, etc., and the risk of the occurrence of the side effects is increased as the administration period of teriparatide is longer. Accordingly, the administration period of the composition containing teriparatide as an active ingredient may vary depending on the age, sex and body weight of a subject to be treated, the particular disease or pathological condition to be treated, the severity of the disease or pathological condition, administration route and the discretion of a prescriber. The determination of the administration period based on these factors is within the level of those skilled in the art and is not limited by the ranges described above.

The composition may be administered immediately after rotator cuff repair. In an aspect of the present disclosure, the term “immediately after” means within 1 second, within 10 seconds, within 1 minute, within 2 minutes, within 5 minutes, within 10 minutes, within 1 hour, within 2 hours, within 4 hours, within 6 hours, within 12 hours or within 24 hours after rotator cuff repair. Since the period between 3 months and 6 months after rotator cuff repair is the most important period for healing, the time when the composition containing teriparatide as an active ingredient is administered is an important factor in healing after rotator cuff repair. The time when the composition ingredient is administered may vary depending on the age, sex and body weight of a subject to be treated, the particular disease or pathological condition to be treated, the severity of the disease or pathological condition, administration route and the discretion of a prescriber, within the ranges described above. In an example of the present disclosure which will be described later, superior healing effect was achieved by administering the composition containing teriparatide as an active ingredient from immediately after rotator cuff repair until 3 months after the repair.

The composition may be administered parenterally depending on purposes. The parenteral administration may be performed by intravenous or intramuscular bolus injection. For the parenteral administration, a formulation for injection such as an isotonic aqueous solution, a suspension, etc. may be prepared according to a method known in the art using a suitable dispersant, wetting agent or suspending agent. For example, a formulation for injection may be prepared by dissolving ingredients in saline or buffer. In addition, the composition may be administered specifically rectally, topically, transdermally, intravenously, intramuscularly, intraperitoneally, subcutaneously, etc. More specifically, it may be administered by subcutaneous injection. The subcutaneous injection may be performed using a syringe suitable for administration of the composition. Specifically, a syringe for administration of teriparatide (Forteo®, Eli Lilly, Indianapolis, Ind.) may be used, although not being limited thereto.

The composition may be for systemic action or topical action of the active ingredient, and may be for systemic or topical administration. Specifically, the composition may be for systemic administration, although not being limited thereto. The composition may be administered abdominally, although not being limited thereto.

The composition may be for reducing, inhibiting or preventing retear after rotator cuff repair. The reduction, inhibition or prevention of retear means reduction, inhibition or prevention of the splitting, ripping, bursting, tearing loose, etc. of a suture site after rotator cuff repair.

The composition may be an injection formulation. The injection formulation may be an injection formulation for subcutaneous injection, although not being limited thereto. In addition, the injection formulation may be for systemic or topical administration. The concentration of the composition may be 1.66-41.67 μg/mL, specifically 1.66 μg/mL or higher, 2 μg/mL or higher, 3 μg/mL or higher, 4 μg/mL or higher, 5 μg/mL or higher, 6 μg/mL or higher, 7 μg/mL or higher, 7.2 μg/mL or higher, 7.4 μg/mL or higher, 7.6 μg/mL or higher, 7.8 μg/mL or higher, 8 μg/mL or higher, 8.1 μg/mL or higher, 8.15 μg/mL or higher, 8.2 μg/mL or higher, 8.25 μg/mL or higher, 8.3 μg/mL or higher, 8.5 μg/mL or higher, 9 μg/mL or higher, 10 μg/mL or higher, 15 μg/mL or higher, 20 μg/mL or higher, 25 μg/mL or higher, 30 μg/mL or higher, 35 μg/mL or higher or 40 μg/mL or higher and 41.67 μg/mL or lower, 40 μg/mL or lower, 30 μg/mL or lower, 25 μg/mL or lower, 20 μg/mL or lower, 15 μg/mL or lower, 10 μg/mL or lower, 9.8 μg/mL or lower, 9.6 μg/mL or lower, 9.4 μg/mL or lower, 9.2 μg/mL or lower, 9 μg/mL or lower, 8.9 μg/mL or lower, 8.8 μg/mL or lower, 8.7 μg/mL or lower, 8.6 μg/mL or lower, 8.5 μg/mL or lower, 8.4 μg/mL or lower, 8.3 μg/mL or lower, 8 μg/mL or lower, 6 μg/mL or lower, 4 μg/mL or lower or 2 μg/mL or lower, although not being limited thereto.

The composition may be a pharmaceutical composition.

The pharmaceutical composition may further contain a pharmaceutically acceptable carrier in addition to the active ingredient teriparatide. The “pharmaceutically acceptable carrier” refers to carrier or a diluent that does not cause significant irritation to an organism and does not abrogate the biological activity and properties of the administered compound. The pharmaceutically acceptable carrier may be a carrier for oral administration such as lactose, starch, a cellulose derivative, magnesium stearate, stearic acid, etc. or a carrier for parenteral administration such as water, a suitable oil, saline, aqueous glucose, glycol, etc. The pharmaceutically acceptable carrier may be used after being mixed with one or more of saline, sterilized water, Ringer's solution, buffered saline, dextrose solution, maltodextrin solution, glycerol and ethanol and, if necessary, other common additives such as a stabilizer, a preservative, an antioxidant, a buffer, a bacteriostat, etc. may be added as an excipient.

In another aspect, the present disclosure provides a kit for healing after rotator cuff repair, which includes the composition for healing after rotator cuff repair. The composition for healing after rotator cuff repair has already been described in detail above.

The kit may include a device for delivering the composition to a subject, and the delivery device may be a syringe. The syringe may be a syringe for administering the composition rectally, topically, transdermally, intravenously, intramuscularly, intraperitoneally, subcutaneously, etc., specifically by subcutaneous injection. The device for subcutaneous injection may be a syringe suitable for administering the composition, or may be a syringe containing the composition. Specifically, a syringe for administration of teriparatide (Forteo®, Eli Lilly, Indianapolis, Ind.) may be used, although not being limited thereto.

The delivery device may be for systemic or topical administration, specifically for systemic administration, although not being limited thereto.

Hereinafter, the constitution and effect of the present disclosure are described more specifically through preparation examples, examples and test examples. However, the following preparation examples, examples and test examples are provided only for helping the understanding of the present disclosure and the category and scope of the present disclosure are not limited by them.

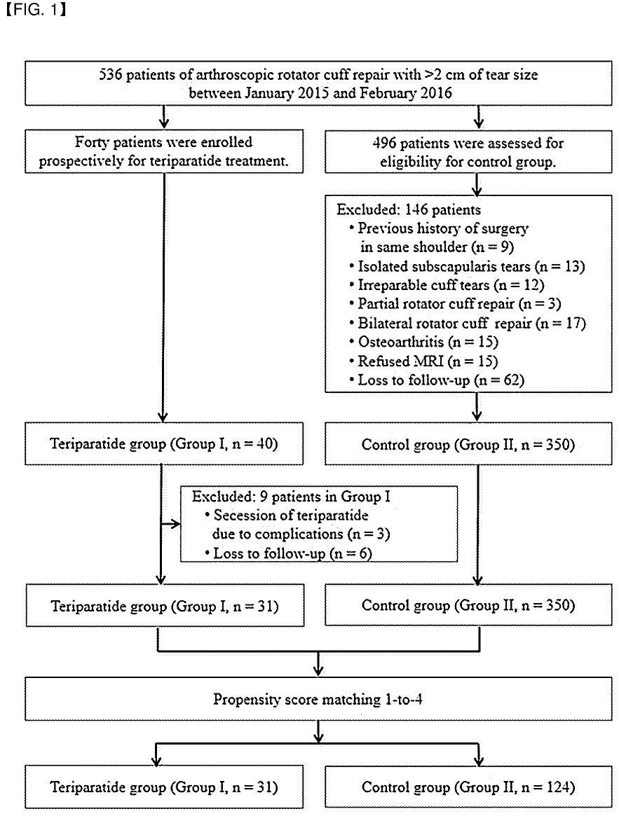

Data collection and all protocols were approved by the Institutional Review Board of Seoul National University Bundang Hospital (IRB No.: B-1802/450-111). According to power analysis, the sample size required 31 patients for a test group and 124 patients for a control group. Assuming a dropout rate of 20%, 40 patients were required for a teriparatide administration group for 3 months surgery (FIG. 1).

[Preparation Example] Preparation of Teriparatide

Teriparatide (Forteo®) used as an active ingredient in the present disclosure was purchased from Eli Lilly (Indianapolis, Ind.).

### [Example 1] Selection of Test Group

For investigation of the healing effect of tear at a suture site of a composition containing teriparatide for arthroscopic rotator cuff repair, a test group was selected as follows.

First, among 613 patients who received arthroscopic rotator cuff repair at Seoul National University Bundang Hospital in Seongnam, Republic of Korea between January 2015 and February 2016, the patients to whom teriparatide would be administered were selected from those who (1) had rotator cuff tear larger than 2 cm in size, (2) received bone mineral density (BMD) measurement before surgery, (3) had radiologic evaluation and functional assessment one year after surgery and (4) could afford the cost of teriparatide treatment. Among them, a total of 40 patients were selected for a teriparatide administration group by excluding those who meet one or more of the following exclusion criteria: (a) rotator cuff tear that cannot be repaired, (b) history of surgery on the same shoulder, (c) history of infectious disease on the same shoulder, (d) history of autoimmune disease, (e) formation of spur 3 mm in size or narrowing of glenohumeral space due to rotator cuff arthropathy or osteoarthritis, (f) history of bisphosphonate administration, (g) contraindication of recombinant human parathyroid hormone, (h) pregnancy, (i) hypercalcemia, (j) severe damage of renal function, (k) metabolic bone disease other than primary osteoporosis, (I) increase of alkaline phosphatase due to unknown cause and (m) radiation therapy for bone tumor or bone metastasis.

Among the 40 patients of the teriparatide administration group, 9 patients were excluded from the test group due to failure of follow-up (n=6), complications including nausea (n=2) or muscle spasm (n=1). Finally, 31 patients were selected for the teriparatide administration test group.

### [Example 2] Administration of Teriparatide to Test Group

20 μg of the teriparatide of Preparation Example was subcutaneously injected every day to the 40 patients of the teriparatide administration group of Example 1 for 3 months after rotator cuff repair. Systemic side effects such as nausea, vomiting, itchiness and muscle spasm after the administration of teriparatide were recorded by a skilled nurse. Based on the result, the data of the 31 patients excluding the 9 patients as described in Example 1 were analyzed. For the 31 patients to which teriparatide was administered, the effect of teriparatide was monitored by measuring serum bone turnover biomarkers including osteocalcin and C-telopeptide (CTX) before and 3 months after the administration.

[Comparative Example] Selection of Control Group

First, among the 613 patients who received arthroscopic rotator cuff repair at Seoul National University Bundang Hospital in Seongnam, Republic of Korea between January 2015 and February 2016, the number of the patients who had rotator cuff tear larger than 2 cm in size was 536. Among the 536 patients, some patients were excluded due to history of surgery on the same shoulder (n=9), isolated subscapularis tear (n=13), rotator cuff tear that cannot be sutured (n=12), partial rotator cuff tear (n=3), rotator cuff tear on both shoulders (n=17), glenohumeral arthritis (n=15), refusal of magnetic resonance imaging (MRI) (n=15) or failure of follow-up (n=62).

Propensity score matching was conducted for 350 patients excluding the above patients. As a result of 1-to-4 matching, 124 patients were selected finally as a control group.

Specifically, k-nearest neighbor matching was conducted using variables including age, sex, surgery at dominant hand, bone mineral density, history of osteoporosis, history of smoking, history of trauma, fatty degeneration of supraspinatus, infraspinatus or subscapularis, tear size of rotator cuff tendon and surgical method. Before the propensity score matching, there was no statistically significant difference between the test group and the control group except the sex of the patients (p<0.001) and surgical method (p<0.001). After the propensity score matching, all the variables were successfully method for the test group, and there was no significant difference in the sex of the patients (p=0.506) and surgical method (p=0.265).

A result of summarizing the variables for the 31 patients of the teriparatide administration test group selected in Example 1 and the 124 patients of the control group of Comparative Example is given in Table 1.

TABLE 1

| Variables | Test group | Control group | p value |
|---|---|---|---|
| Number of patients | 31 | 124 | |
| Age (years) | 64.3 ± 7.4 | 63.9 ± 8.0 | 0.816 |
| Sex (male/female) | 11/20 | 53/71 | 0.506 |
| Onset (months, (range)) | 30.8 (2-120) | 25.1 (1-120) | 0.465 |
| Surgery at dominant hand (yes/no) | 21/10 | 88/46 | 0.413 |
| Mean follow-up (F/U) period (months, (range)) | 26.0 (24-30) | 27.6 (25-31) | 0.561 |
| Bone mineral density (T-score) | −1.6 ± 1.1 | −1.6 ± 1.3 | 0.959 |
| Osteoporosis (yes/no) | 10/21 | 40/84 | 0.975 |
| History of smoking (yes/no) | 3/28 | 12/112 | 0.672 |
| History of trauma (yes/no) | 19/12 | 72/52 | 0.988 |
| Radiological evaluation (MRI/CTA) | 31/0 | 120/4 | 0.228 |
| Preoperative fatty degeneration | | | |
| Supraspinatus | 2.2 ± 0.9 | 2.1 ± 0.9 | 0.829 |
| Infraspinatus | 1.4 ± 0.6 | 1.3 ± 0.8 | 0.860 |
| Teres minor | 1.0 ± 1.2 | 0.7 ± 0.7 | 0.234 |
| Subscapularis | 1.3 ± 0.6 | 1.2 ± 0.9 | 0.599 |
| Global fatty degeneration index | 1.3 ± 0.6 | 1.3 ± 0.5 | 0.785 |
| Tear size (cm) | | | |
| Anteroposterior (AP) dimension | 2.5 ± 0.8 | 2.5 ± 0.7 | 0.867 |
| Retraction | 2.3 ± 0.6 | 2.4 ± 0.8 | 0.775 |
| Rotator cuff tear range (%) | | | 0.664 |
| SSP only | 15 (48.4) | 64 (51.6) | |
| SSP + ISP | 6 (19.4) | 28 (22.6) | |
| SSP + SSC | 8 (25.8) | 24 (19.4) | |
| SSP + ISP + SSC | 2 (6.5) | 8 (6.5) | |
| Combined lesion | | | |
| Subscapular muscle tear | 10 | 32 | 0.374 |
| Acromioclavicular (AC) arthritis | 11 | 35 | 0.769 |
| Superior labrum anterior-to-posterior | 21 | 52 | 0.141 |
| (SLAP) lesion | | | |
| Biceps tear | 23 | 53 | 0.350 |
| Surgery (DR/SR) | 17/14 | 75/49 | 0.265 |
| Tenodesis/tenotomy | 8/15 | 15/38 | 0.561 |
| Distal clavicle resection | 2 | 9 | 0.153 |
| Operation time (minutes) | 116.3 ± 17.5 | 106.8 ± 11.8 | 0.698 |

*F/U: follow-up, MRI: magnetic resonance imaging, CTA: computed tomography arthrography, AP: anteroposterior, SSP: supraspinatus, ISP: infraspinatus, SSC: subscapularis, AC: acromioclavicular, SLAP: superior labrum anterior-posterior, DR: double-row suture bridge, SR: single-row.

The 124 patients of the control group were not administered with teriparatide unlike the patients of the test group.

Surgery and Rehabilitation of Patients of Test Group and Control Group

All orthopedic surgeries of the patients of the test group and the control group were conducted by senior orthopedists under general anesthesia. After performing debridement on the edge of torn rotator cuff, tear size and degree of retraction were measured with a probe. Then, a bleeding surface was created on the greater tuberosity of humerus to improve tendon-to-bone healing. The cortex was not completely removed to maximize resistance and to reduce loosening of a structure anchor. Double-row suture repairs were performed because the tear size of all the patients was larger than 2 cm. If the torn tendon was not pulled to the original position due to severe retraction, single-row repair was performed to reduce the tension of the treated tendon. The patients were asked to wear abduction braces for 5-6 weeks after the surgery. Shrugging shoulders, active elbow bending/stretching, active elbow supination/pronation and active motion of hands and wrists were allowed immediately after the surgery. Passive motion of forward elevation was conducted immediately after the surgery. After removing the abduction braces, the patients were trained for active and passive shoulder exercise. After the range of shoulder motions were recovered, shoulder muscle strengthening was started at 9-12 weeks after the surgery. All athletic activities were allowed 6 months after the surgery.

Statistics

According to a research which showed that retear rate was significantly higher in patients with a tear size larger than 2 cm (34.2%) than in patients with a tear size of 2 cm or smaller (10.6%, p<0.001) (Park J S, Park H J, Kim S H, Oh J H. Prognostic Factors Affecting Rotator Cuff Healing After Arthroscopic Repair in Small to Medium-sized Tears. The American journal of sports medicine. 2015; 43: 2386-2392), the minimum sample size required for a priori analysis was 31 for the administration group (test group) and 124 for the control group. The test group showed significant decrease in retear rate with a statistical power of 0.80 (α=5%).

Propensity score matching was performed using PASS 11 (version 11.0, Kaysville, Utah). Other statistical analysis was performed using the SPSS software (version 21.0, IBM Corp., Armonk, N.Y.). When the data showed a normal distribution, independent t-test was used to assess the difference between the test group and the control group and paired t-test was used to compare the variables before and after the surgery. Other nonparametric variables were processed by χ² analysis and Fisher's exact test.

[Test Example 1] Anatomical Assessment

The anatomical assessment on healing after rotator cuff repair of the test group of Example 1 and the control group of Comparative Example was conducted based on radiological assessment.

Specifically, the treatment of rotator cuff tendon was assessed at 1 year or later after the surgery by MRI or computed tomography arthrography (CTA) using a contrast agent (Farin P U, Kaukanen E, Jaroma H, Vaatainen U, Miettinen H, Soimakallio S. Site and size of rotator-cuff tear. Findings at ultrasound, double-contrast arthrography, and computed tomography arthrography with surgical correlation. Investigative radiology. 1996; 31:3 87-394; Ostor A J, Richards C A, Tytherleigh-Strong G, et al. Validation of clinical examination versus magnetic resonance imaging and arthroscopy for the detection of rotator cuff lesions. Clinical rheumatology. 2013; 32: 1283-129121, 22). Fatty degeneration of rotator cuff muscle was assessed according to Goutallier's global fatty degeneration index (GFDI), etc. (Goutallier D, Postel J M, Gleyze P, Leguilloux P, Van Driessche S. Influence of cuff muscle fatty degeneration on anatomic and functional outcomes after simple suture of full-thickness tears. Journal of shoulder and elbow surgery. 2003; 12: 550-554). CTA was performed when MRI was unavailable due to cost, presence of a pacemaker or other contraindications. All the patients of the test group of Example 1 were radiologically assessed by MRI. For the patients of the control group of Comparative Example, MRI was performed for 96.8% (120 out of 124 patients) and CTA was performed for the remaining 4 patients. The healing of tendon was radiologically assessed by a musculoskeletal radiologist with a career of 14 years, who was unaware of the study, according to Sugaya's classification (Sugaya H, Maeda K, Matsuki K, Moriishi J. Functional and structural outcome after arthroscopic full-thickness rotator cuff repair: single-row versus dual-row fixation. Arthroscopy: the Journal of Arthroscopic & Related Surgery: official publication of the Arthroscopy Association of North America and the International Arthroscopy Association. 2005; 21: 1307-1316). Types IV and V were regarded as retear. The result is shown in Table 2.

TABLE 2

| Variables | Test group | Control group | p value |
|---|---|---|---|
| Retear rate | 16.1% (5 out of 31) | 33.9% (42 out of 124) | 0.037 |
| Postoperative | | | |
| fatty | | | |
| degeneration | | | |
| Supraspinatus | 1.9 ± 0.7 | 2.0 ± 0.8 | 0.387 |
| Infraspinatus | 1.2 ± 0.7 | 1.3 ± 0.9 | 0.426 |
| Teres minor | 0.7 ± 1.3 | 0.6 ± 1.0 | 0.736 |
| Subscapularis | 1.1 ± 0.7 | 1.2 ± 1.0 | 0.318 |
| Global fatty | 1.1 ± 0.7 | 1.3 ± 0.6 | 0.249 |
| degeneration | | | |
| index | | | |

As seen from Table 2, the retear rate was 16.1% (5/31) for the test group, and 33.9% (42/124) for the control group (p=0.037). There was no significant difference between the test group and the control group in global fatty degeneration index (p=0.249) or fatty degeneration after rotator cuff repair (SSP, ISP, TM and SSC, p=0.387, 0.426, 0.736 and 0.318, respectively).

[Test Example 2] Assessment of Range of Motions (ROMs), Functional Outcomes and Isokinetic Muscle Performance Test (IMPT)

The range of motions (ROMs) and functional outcomes of the test group of Example 1 and the control group of Comparative Example were assessed. Specifically, all the patients of the test group and the control group were assessed for shoulder range of motion (ROMs), American Shoulder and Elbow Surgeons (ASES) score, Constant score and simple shoulder test (SST) before surgery and 6 months and 1 year after the surgery. The assessment was conducted by clinical researchers who were unware of the patients' information.

In addition, for evaluation of the effect of teriparatide on muscle power, isokinetic muscle performance test (IMPT) was performed for the test group of Example 1 and the control group of Comparative Example (Biodex System 3 Pro, Biodex Medical System, Inc., Shirley, N.Y.). The test is correlated with the condition of the rotator cuff. The difference in peak torque was compared with that of the uninjured shoulder. A larger value means that the peak muscle power is insufficient to that of uninjured shoulder.

The result of ROMs, functional outcomes and IMPT assessment is shown in Table 3.

TABLE 3

| Variables | Test group | Control group | p value |
|---|---|---|---|
| Range of motions (ROMs) | | | |
| Forward elevation (°) | 159.6 ± 18.9 | 158.9 ± 23.2 | 0.862 |
| External rotation (°) | 62.9 ± 13.6 | 67.4 ± 15.0 | 0.142 |
| Internal rotation | T8.4 ± 1.7 | T8.2 ± 1.4 | 0.586 |
| ASES score | 96.1 ± 8.2 | 92.7 ± 12.4 | 0.203 |
| Constant score | 70.1 ± 5.5 | 69.5 ± 8.0 | 0.439 |
| Simple Shoulder Test | 11.0 ± 1.9 | 10.2 ± 2.9 | 0.185 |
| Isokinetic muscle | | | |
| performance test | | | |
| (IMPT, %) | | | |
| Abduction | 12.8 ± 35.5 | 17.5 ± 24.2 | 0.574 |
| Adduction | −0.2 ± 18.5 | 0.6 ± 17.8 | 0.564 |
| External rotation | 13.6 ± 21.8 | 15.1 ± 24.4 | 0.791 |
| Internal rotation | 5.6 ± 13.8 | 5.6 ± 17.6 | 0.965 |

As seen from Table 3, in range of motions (ROMs) assessment, the angle of forward elevation was improved from 145.7±20.1° to 159.6±18.9°, the angle of external rotation was improved from 53.5±10.4° to 62.9±13.6° and the internal rotation was improved from T10.1±2.6 to T8.4±1.7, for the test group (p=0.006, 0.008 and 0.001). For the control group, the angle of forward elevation was improved from 149.9±30.1° to 158.9±23.2°, the angle of external rotation was improved from 52.6±16.1° to 67.4±15.0° and the internal rotation was improved from T9.7±2.4 to T8.2±1.4 (p=0.002, <0.001 and <0.001, respectively). However, no statistical difference was observed in the improvement of ROM in the final follow-up monitoring between the two groups (p>0.05).

The functional outcomes were also improved significantly for the test group in ASES score, Constant score and all performance tests including SST (ASES score, Constant score and SST improved from 53.2±17.8 to 96.1±8.2, from 51.8±9.4 to 70.1±5.5 and from 3.9±3.0 to 11.0±1.9, respectively, p<0.001). Also, for the control group, ASES score, Constant score and SST were improved from 52.5±17.8 to 92.7±12.4, from 53.3±14.1 to 69.5±8.0 and from 4.3±3.1 to 10.2±2.9, respectively (p<0.001). There was no difference in functional scores in the final follow-up monitoring between the two groups.

As a result of the IMPT, both the test group and the control group showed improved muscle power at 1 year after the surgery, and there was no significant difference between the test group and the control group (p>0.05).

[Test Example 3] Effect of Teriparatide on Bone Turnover Biomarkers

For evaluation of the effect of the composition containing teriparatide on bone mineral density (BMD), BMD was measured for all the patients who received arthroscopic rotator cuff repair. The BMD was measured before surgery by dual-energy X-ray absorptiometry (DXA, Lunar Prodigy, enCORE version 8.8, GE Medical Systems, Milwaukee, Wis.). The lowest T scores of the proximal femur and the lumbar were recorded. The score at the Ward's triangle region of the proximal femur was excluded.

As a result, for the test group of Example 1 to which the composition containing teriparatide of Preparation Example was administered, the level of osteocalcin was increased from 18.6±6.7 ng/mL to 27.9±15.4 ng/mL at 3 months after the surgery (p=0.003), and the level of C-telopeptide (CTX) was increased from 0.4±0.2 ng/mL before the surgery to 0.5±0.3 ng/mL at 3 months after the surgery (p=0.181).

Accordingly, it was confirmed that teriparatide is effective for tendon-to-bone healing in the patients who received rotator cuff repair, particularly the patients with a tear size larger than 2 cm. In particular, it was confirmed that teriparatide is effective in reducing retear rate in the patients who received rotator cuff repair and have a tear size larger than 2 cm.

Meanwhile, the patients who were administered with teriparatide in the present disclosure showed fewer side effects than reported in other researches, which may be related with the short administration period of 3 months. According to a previous report, the expression of bone formation markers including osteocalcin was increased in the early stage of teriparatide treatment and the expression of resorption markers such as CTX was observed later (Finkelstein J S, Wyland J J, Lee H, Neer R M. Effects of teriparatide, alendronate, or both in women with postmenopausal osteoporosis. J Clin Endocrinol Metab 2010; 95: 1838-1845). Therefore, the increased expression of osteocalcin among bone turnover markers at 3 months after the surgery confirms that teriparatide was successfully administered to the patients.

### INDUSTRIAL APPLICABILITY

The present disclosure relates to a composition for healing after rotator cuff repair, more specifically to a composition containing teriparatide as an active ingredient for healing of tear at the suture site after rotator cuff repair. The composition exhibits a tendon-to-bone healing effect when administered to patients, especially those with a tear size larger than 2 cm, for treatment of rotator cuff and is effective in lowering retear rate, and thus can lead to improved healing after rotator cuff repair.

1. A method for healing after rotator cuff repair,
which comprises administering a composition comprising teriparatide, an isomer thereof, a pharmaceutically acceptable salt thereof, a hydrate thereof or a solvate thereof to a subject in need of healing after rotator cuff repair, wherein,
the healing after rotator cuff repair is healing of tear at the suture site after rotator cuff repair, and
the composition is administered to a patient having a rotator cuff tear size of larger than 2 cm.

2. The method according to claim 1, wherein the composition is administered for 2-24 weeks after rotator cuff repair.

3. The method according to claim 1, wherein the composition is administered by subcutaneous injection.

4. The method according to claim 1, wherein the composition is for systemic or topical administration.

5. The method according to claim 1, wherein the composition is an injection formulation.

6. The method according to claim 5, wherein the concentration of the composition is 1.66-41.67 μg/mL.

7. The method according to claim 1, wherein the composition is administered for reducing retear after rotator cuff repair.

8. The method according to claim 1, wherein the composition is administered immediately after rotator cuff repair.

9-11. (canceled)
